(19) United States
(12) Patent Application Publication
(10) Pub. No.: US 2011/0118612 A1
(43) Pub. Date: May 19, 2011

(54) Valved Catheter with Integrated Pressure Measurement Capabilities

(75) Inventor: Stephen Miller, Queensbury, NY (US)

(73) Assignee: Navilyst Medical, Inc., Marlborough, MA (US)

(21) Appl. No.: 12/621,193

(22) Filed: Nov. 18, 2009

(51) Int. Cl. A61B 5/0215; A61M 39/22

(52) U.S. Cl. 600/486; 604/537

(57) ABSTRACT

Valve assemblies and valved catheters including pressure sensors, and related methods for treating patients, are disclosed. The valve assembly includes a valve member extending across a first lumen and includes a planar flexible member with an internal slit acting as a valve. The valve assembly further includes a pressure sensor located distally from the valve member and in fluid communication with the first lumen.

### FIELD OF THE INVENTION

The present invention relates to valved catheters, and more particularly, to valved catheters that include integrated pressure measuring capabilities.

### BACKGROUND

There are a number of implantable medical devices used for the repeated and prolonged access to a patient's vascular system or other bodily conduits. Such devices include peripherally-inserted central catheters (“PICC's”), central venous catheters (“CVC's”), dialysis catheters, implantable ports, and midline infusion catheters. These devices are typically implanted into a patient for an extended period of time to allow for multiple treatments, such as the delivery of therapeutic agents or dialysis treatments. Use of such devices eliminates the need for multiple placements of single-use devices, thus reducing the risk of infection and placement complications, and reducing the overall cost of patient care. Examples of such implantable medical devices include Vaxcel® PICC's and ports, Xcela® PICC's and ports, and Vaxcel® Plus Chronic Dialysis catheters (all from Navilyst Medical, Inc., Marlborough, Mass.).

Because the aforementioned devices remain in a patient's body for an extended period of time, it is common practice to seal their proximal ends between uses to prevent blood loss and infection. Such a seal may be created with the use of a simple clamp placed on the catheter line, or more recently, with the use of an in-line valve such as that found in the Vaxcel® PICC with PASV® Valve Technology (Navilyst Medical, Inc., Marlborough, Mass.) and described in U.S. Pat. Nos. 5,205,834 and 7,252,652, which are incorporated herein by reference. In-line valves are pressure activated such that they open to allow for fluid to be delivered to a patient upon the application of some threshold pressure, above which the valve will open, and below which the valve remains closed. These valves are believed to represent improved performance over simple clamps and result in fewer patient complications and infections.

Computed tomography (CT) is increasingly used as a imaging technique for long-term medical patients. Many CT techniques make use of contrast agents to yield high quality images, thus requiring that the contrast agents be administered to the patient prior to the CT imaging. For patients that already have an implanted device that provides access to the vasculature or organ desired to be imaged, it is desirable to use the existing implanted device as a means for administering the contrast agent rather than to make another incision or introduce another catheter line into the patient for this purpose. Given the usual quantity of contrast agent and the short time frame over which it should be administered, however, it is necessary to inject the contrast agent at a relatively high flow rate, such as 5 cc/sec. Not all implantable devices are configured to deliver fluid at this flow rate, or to handle the pressures associated therewith. Some commercial products have recently been developed that use dimensions, configurations, and/or materials that render them suitable for such so-called “power” injections. An example is the Xcela® Power Injectable PICC (Navilyst Medical, Marlborough, Mass.). In order to use implantable devices that are power injectable and make use of in-line valves, it is necessary to ensure that the valve portion of these devices are capable of handling the flow rates and pressures associated with power injection.

During patient treatment with a PICC or similar device, it is often desirable to measure the patient's blood pressure locally at the site of device implantation. When the PICC or similar device is non-valved, blood pressure measurements are made by inserting an elongated blood pressure measuring tool though the PICC lumen to contact blood and measure pressure. In the case of valved devices, however, it is not possible to take blood pressure measurements in this manner because of the risk of damaging the valve during the insertion and withdrawal of the pressure measurement tool.

### SUMMARY OF THE INVENTION

In one aspect, the present invention relates to a valve assembly for use with a catheter, wherein the valve assembly includes a blood pressure sensor.

In another aspect, the present invention relates to a catheter system that includes a catheter and a valve assembly that includes a blood pressure sensor.

In another aspect, the present invention relates to methods of treating patients and monitoring their blood pressure by using the valve assemblies and/or valved catheters of the present invention.

In yet another aspect, the present invention relates to kits that include a valved catheter that includes a blood pressure sensor.

In one embodiment, the present invention is a valve assembly used in conjunction with a catheter. The valve assembly includes a proximal end, a distal end in contact with the catheter, and a first lumen in fluid communication with the catheter. The valve assembly includes a valve member extending across the first lumen and comprises a planar flexible member comprising first and second valve portions separated from one another by an internal slit, the first and second valve portions moving, when subjected to a fluid pressure of at least a predetermined threshold level, to a first open position so that material may flow distally through the valve member, through the first lumen, and into the catheter. The first and second valve portions remain substantially closed at all times when a fluid pressure exerted thereagainst is less than the threshold level to substantially prevent flow through the valve member. The valve assembly further includes a pressure sensor located distally from the valve member.

In another embodiment, the present invention is a catheter or other implantable medical device that includes the valve assembly of the present invention.

In yet another embodiment, the present invention is a kit that includes the valve assembly, catheter, or other implantable medical device of the present invention.

### DESCRIPTION OF THE DRAWINGS

FIG. 1 is an example of a prior art peripherally-inserted central catheter.

FIG. 2 is an exploded view of an example of a prior art valve assembly that is used in central catheters.

FIG. 3 is a perspective view of a catheter system, in accordance with an embodiment of the present invention.

FIG. 4 is a cross-sectional view of a valve assembly, according to an embodiment of the present invention in which a pressure sensor is placed in an “on” configuration.

FIG. 5 is a cross-sectional view of a valve assembly, according to an embodiment of the present invention in which a pressure sensor is placed in an “off” configuration.

FIG. 6 is a perspective view of a catheter system, in accordance with an embodiment of the present invention.

### DETAILED DESCRIPTION OF THE PREFERRED EMBODIMENTS

The present invention relates to devices and methods in which blood pressure measurements may be taken through a valved catheter. Whereas blood pressure measurements may be taken using elongated tools inserted through conventional non-valved catheters, such a method runs the risk of damaging any internal valve if performed within a valved catheter. The present invention avoids the risk of damaging the valve within valved catheters by using a pressure sensor that is integral with the catheter and does not require the insertion of a pressure measurement tool into the catheter lumen.

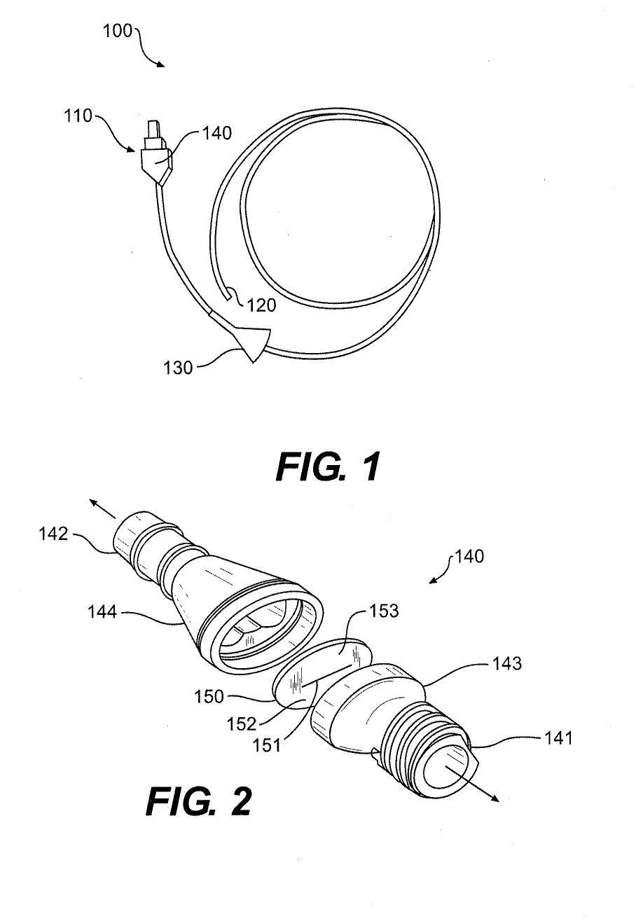

Examples of medical devices that are useful in the present invention include peripherally-inserted central catheters (“PICC's”), central venous catheters (“CVC's”), dialysis catheters, implantable ports, and midline infusion catheters that include internal valves. By way of example, FIG. 1 shows a PICC that makes includes an internal valve. As shown in FIG. 1, PICC 100 includes a proximal end 110 that, when in use, extends outside of a patient, a distal end 120 that is implanted into the patient's vasculature system, a suture wing 130 for attaching to the patient, and a valve assembly 140 connected to proximal end 110. The distal end 120 (shown curled in FIG. 1) up to the suture wing 130 remains implanted in the patient for an extended period of time for the repeated delivery of therapeutic agents. The in-line valve assembly 140 is used, for example, to seal the PICC so that blood does not flow into the PICC when left in place, and contaminants do not enter the PICC.

An example of a valve assembly 140 that is useful for use in PICCs and other devices of the present invention is described in U.S. Pat. No. 7,252,652, which is incorporated herein by reference. FIG. 2 shows an exploded view of such an assembly, which includes proximal end 141, distal end 142, male housing portion 143, female housing portion 144, and planar, flexible valve member 150. In use, the proximal end 141 is connected to a syringe, IV line, or the like to inject or otherwise deliver fluid to a patient. Such fluids include, for example, therapeutic agents and contrast agents. The distal end 142 is attached as part of a PICC (as shown in FIG. 1) or other suitable device. In the embodiment shown in FIG. 2, the male and female housing portions 143, 144 fit together to house the valve member 150. The valve member 150 includes a slit 151 that is “internal” such that it does not extend to any edge of the valve member 150. The valve member includes first and second valve portions 152, 153 on either side of slit 151. When subjected to a fluid exerted in the distal direction characterized by a pressure of at least a predetermined threshold level, the first and second valve portions 152, 153 move to open the slit in the distal direction so that the fluid may flow distally through the valve member 150 and out the distal end 142 of the housing 140. At pressures lower than this threshold level, the slit remains closed so as to substantially prevent the flow of fluid therethrough. For example, the valves of the present invention remain closed during normal increases in central venous pressure. Whereas the present invention is illustrated as having a single slit 151 within the valve member 150, the invention includes valve members 150 that comprise multiple slits 151.

In a preferred embodiment, the valve used in the valve assemblies and catheters of the present invention is a two-way valve such that, in addition to opening in a distal direction, it also opens in a proximal direction when subjected to a fluid exerted in the proximal direction characterized by a pressure of at least a predetermined threshold level which may be the same or different from the threshold level required to open the valve in the distal direction. Such two-way valves are useful, for example, to aspirate blood or other bodily fluids for sampling or other purposes. The valves of the present invention are also preferably useful for administering power injections of contrast agents.

Suitable materials used to form the valve member 150 include, for example, silicone, rubber, and other elastomeric materials. These materials are formed into the shape of the valve member 150 using any suitable manufacturing technique such as, for example, liquid injection molding, rubber compression molding, and calendaring followed by die cutting.

It is often desirable to measure the blood pressure, and more specifically the central venous pressure (CVP), of patients that have an implanted PICC or similar device. The CVP represents the pressure of the blood in the thoracic vena cava, and can provide important information such as the volume of blood circulation and how well the heart is pumping. To measure the CVP, it has previously been necessary to introduce a catheter through a patient's neck or chest to a point near the right atrium. The catheter is then connected to a small diameter water column which is calibrated to indicate the CVP, or an elongated pressure measuring device is inserted through the catheter to make the measurement. These methods are not possible with a valved catheter such as the PICC shown in FIGS. 1 and 2 because the CVP may be less than the pressure required to open the valve proximally, and the insertion of an elongated pressure measurement device through the valve could result in damage to the valve.

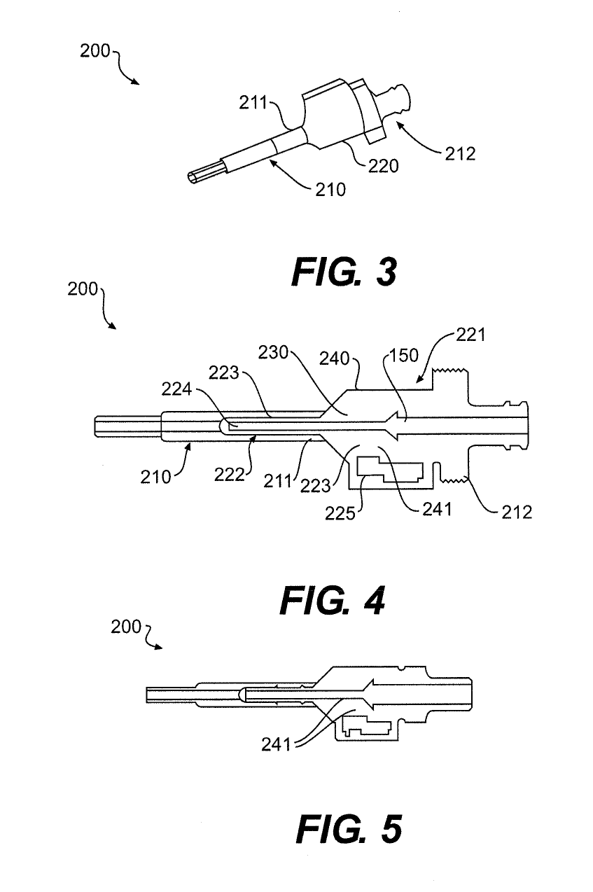

To facilitate the measurement of blood pressure, and particularly CVP, using valved catheters, the inventor has conceived a new catheter system 200 as shown in FIG. 3. Catheter system 200 includes a catheter tube 210 that is insertable into the vasculature of a patient and having a proximal end 211 intended to remain outside of a patient's body, and a valve assembly 220 at the proximal end 211 of catheter tube 210. The catheter system 200 is optionally adapted at its proximal end 212 to receive an infusion tube, syringe, or the like, and may be configured as a luer fitting as shown in FIG. 3.

As shown in FIG. 4, the valve assembly 220 includes a proximal end 221, a distal end 222 in contact with the catheter, and a first lumen 224 extending between the proximal and distal ends 221, 222. The distal end 222 of the valve assembly 220 is preferably insertable into the proximal end 211 of the catheter tube 210, and optionally includes radial protrusions 223 configured to firmly secure the valve assembly 220 to the catheter tube 210. The first lumen 224 is in fluid communication with the catheter tube 210. The valve assembly 220 further includes a valve member 150 extending across the first lumen 224, the valve member comprising a planar flexible member with first and second valve portions separated from one another by an internal slit, as previously described. The valve assembly 220 further includes a pressure sensor 225 located distally relative to the valve member 150. The pressure sensor 225 is in fluid communication with the first lumen 224 so that it can measure the pressure of fluid, such as blood, within the first lumen 224.

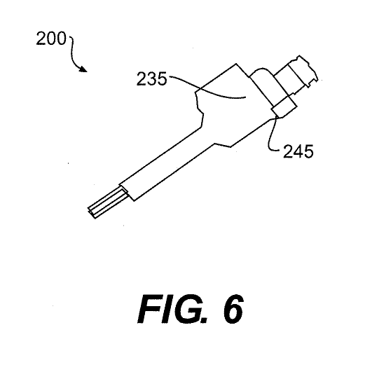

In one embodiment, the valve assembly 220 comprises valve housing 230 and sensor housing 240 portions that are connected to each other, as shown in FIG. 4. In a preferred embodiment, the sensor housing 240 is rotatable about the valve housing 230. Also preferred is the use of a second lumen 241 that is bisected by the sensor housing 240 and the valve housing 230, with the second lumen 241 extending between the pressure sensor 225 and the first lumen 224. A pressure seal 223, such as a silicone O-ring, is optionally placed at the location where the second lumen 241 is bisected by the sensor and valve housings 240, 230. In the embodiment in which the sensor housing 240 is rotatable about the valve housing 230, it is moveable from a position in which the second lumen 241 is in fluid communication with the first lumen 224 such that the sensor is in an “on” position, as shown in FIG. 4, to a position in which the second lumen 241 (or at least the portion of the second lumen that is part of the sensor housing 240) is not in fluid communication with the first lumen 224 such that the sensor is in an “off” position, as shown in FIG. 5. Also for embodiments in which the sensor housing 240 is rotatable about the valve housing 230, it is preferred that the housings 240, 230 include a geometry such as respective first and second contacting elements 245, 235 as shown in FIG. 6, which are configured to make contact during the rotation to indicate that the housings 240, 230 are in a position in which the second lumen 241 is in fluid communication with the first lumen 224.

The embodiment of the present invention in which the sensor housing 240 is rotatable about the valve housing 230 is particularly well-suited for valved catheters that are used for power injections, as previously described. During such power injections, the sensor housing 240 is rotated to a position in which the second lumen 241 (or at least the portion of the second lumen that is part of the sensor housing 240) is not in fluid communication with the first lumen 224, as shown in FIG. 5. By so placing the sensor in its “off” position, the risk of damaging the sensor by high pressure power injections is minimized. After the power injection is complete, the sensor housing 240 may be rotated back so that the sensor is returned to an “on” position.

It should be appreciated that although the sensor and valve housings 240, 230 are described herein to be moveable with respect to each other by rotation, the present invention includes any other arrangements, in accordance with preferred embodiments, in which the housings are moveable with respect to each other to result in the pressure sensor 225 being out of fluid communication with the first lumen 224. For example, the sensor housing 240 may be axially slidable with respect to the valve housing 230.

The pressure sensor 225 is any suitable sensor as is known in the art, such as a “Wheatstone Bridge” type sensor. Such sensors contain a pressure diaphragm that is one plate of a capacitor that changes its value under pressure-induced displacement. Pressure sensing is conducted by measuring the difference in pressure of the two sides of the diaphragm. As is known in the art, this type of pressure sensor may consist of a micro-machined silicon diaphragm with piezoresistive strain gauges diffused into it, fused to a silicon or glass backplate. The strain gauges act as resistive elements, and under the press-induced strain, the resistive values change. The resistors are connected as a Wheatstone Bridge, the output of which is directly proportional to the pressure. An example of a pressure sensor that may be suitable for use with the current invention is GE NovaSensor's NPC-100 (GE Novasensor, Inc., Fremont, Calif.). The pressure sensor 225 includes an electrical connection (not shown) to a voltage source to power the pressure sensor, and a data connection (not shown) to a user interface to provide or display pressure information.

When the pressure sensor 225 is in fluid communication with the first lumen 224, it can be used to measure the blood pressure, such as the CVP, of a patient. In comparison to conventional valved catheters, the use of the catheter system 200 of the present invention avoids the need for the removal of the valved catheter and the introduction of additional non-valved catheters to facilitate the taking of blood pressure measurements.

Components of the valve assembly 220 are manufactured using known techniques, such as injection molding. The valve assembly 220 may be sold as a separate component intended to be inserted into a catheter tube, or as part of a fully assembled catheter system. The valve assemblies 220 and catheter systems 200 of the present invention are used in suitable methods for treating patients and monitoring their blood pressure, such as their CVP, by using the valve assemblies and/or valved catheters of the present invention. For example, the PICCs of the present invention are inserted into the venous system of a patient using known techniques, such as those used for the placement of Vaxcel® PICCs with PASV® Valve Technology (Navilyst Medical, Inc., Marlborough, Mass.). The CVP of the patient may be measured and monitored using the pressure sensor integrated within the catheter system. In the event that pressure measurements are temporarily not desired or power injections of contrast agent or the like are introduced through the catheter, the sensor is moved into an “off” position during, such as by rotation of a sensor housing as described herein for preferred embodiments. The use of the catheter for its intended purposes, such as the introduction of medications or the withdrawal of blood, can take place regardless of whether the sensor is in its “off” or “on” position.

It will be apparent to those skilled in the art that various modifications and variations can be made in the structure and methodology of the present invention. Thus, it is intended that the present invention cover the modifications and variations of this invention provided that they come within the scope of the appended claims and their equivalents.

I claim:

1. A valve assembly for use with a catheter comprising:
a proximal end, a distal end in contact with the catheter, and a first lumen extending between said proximal and distal ends, said first lumen being in fluid communication with the catheter,
a valve member extending across said first lumen, said valve member comprising:
a planar flexible member comprising first and second valve portions separated from one another by an internal slit,
the first and second valve portions moving, when subjected to a fluid pressure of at least a predetermined threshold level, to a first open position so that material may flow distally through said valve member, through said first lumen, and into the catheter,
the first and second valve portions remaining substantially closed at all times when a fluid pressure exerted thereagainst is less than the threshold level to substantially prevent flow through said valve member; and
a pressure sensor located distally from said valve member.

2. The valve assembly of claim 1, further comprising a valve housing and a sensor housing, wherein said valve housing contains said first lumen and said valve member, and said sensor housing comprises said pressure sensor and a second lumen extending between said pressure sensor and said first lumen.

3. The valve assembly of claim 2, wherein said sensor housing is rotatable about said valve housing.

4. The valve assembly of claim 3, wherein when said sensor housing is rotated about said valve housing, it is moveable from a position in which said second lumen is in fluid communication with said first lumen to a position in which said second lumen is not in fluid communication with said first lumen.

5. The valve assembly of claim 4, wherein said sensor housing comprises a first contacting element and said valve housing comprises a second contacting element, said first and second contacting elements being configured to make contact during the rotation of said sensor housing about said valve housing to indicate that said sensor housing is in said position in which said second lumen is in fluid communication with said first lumen.

6. The valve assembly of claim 2, further comprising a pressure seal extending across said second lumen.

7. The valve assembly of claim 1, wherein said distal end of said assembly is insertable into the catheter.

8. The valve assembly of claim 1, further comprising an electrical connection between said pressure sensor and a voltage source.

9. The valve assembly of claim 1, further comprising a data connection between said pressure sensor and a user interface.

10. A valve assembly for use with a catheter comprising:
a valve housing and a sensor housing;
said valve housing comprising:
a proximal end, a distal end in contact with the catheter, and a first lumen extending between said proximal and distal ends, said first lumen being in fluid communication with the catheter,
a valve member extending across said first lumen, said valve member comprising a planar flexible member comprising first and second valve portions separated from one another by an internal slit,
the first and second valve portions moving, when subjected to a fluid pressure of at least a predetermined threshold level, to a first open position so that material may flow distally through said valve member, through said first lumen, and into the catheter,
the first and second valve portions remaining substantially closed at all times when a fluid pressure exerted thereagainst is less than the threshold level to substantially prevent flow through said valve member;
said sensor housing comprising:
a pressure sensor located distally from said valve member, and
a second lumen extending between said pressure sensor and said first lumen.

11. The valve assembly of claim 10, wherein said sensor housing is rotatable about said valve housing.

12. The valve assembly of claim 11, wherein when said sensor housing is rotated about said valve housing, it is moveable from a position in which said second lumen is in fluid communication with said first lumen to a position in which said second lumen is not in fluid communication with said first lumen.

13. The valve assembly of claim 12, wherein said sensor housing comprises a first contacting element and said valve housing comprises a second contacting element, said first and second contacting elements being configured to make contact during the rotation of said sensor housing about said valve housing to indicate that said sensor housing is in said position in which said second lumen is in fluid communication with said first lumen.
